(19) United States
(12) Patent Application Publication
(10) Pub. No.: US 2023/0060997 A1
(43) Pub. Date: Mar. 2, 2023

(54) RADIONUCLIDE-LHRH CONJUGATES FOR DIAGNOSIS OF REPRODUCTIVE CANCERS

(71) Applicant: EASTERN KENTUCKY UNIVERSITY, Richmond, KY (US)

(72) Inventors: Margaret NDINGURI, Richmond, KY (US); Lindsay CORMIER, Richmond, KY (US)

(21) Appl. No.: 17/792,571

(22) PCT Filed: Jan. 14, 2021
(86) PCT No.: PCT/US2021/013362

§ 371 Date: Jul. 13, 2022

Related U.S. Application Data
(60) Provisional application No. 62/961,107, filed on Jan. 14, 2020.

(51) Int. Cl. A61K 51/08

(52) U.S. Cl. CPC A61K 51/088

(57) ABSTRACT

The present disclosure concerns luteinizing hormone receptor hormone (LHRH) compositions that can selectively bind to an LHRH receptor. Through the incorporation of radionuclides in the LHRH compositions, selective radiolabeling is made possible, allowing for identification of cells over-expressing the LHRH receptor, which is often a facet of an abnormally growing cell or a tumorigenic cell. The LHRH compositions can therefore provide for improved detection and monitoring of cancers associated with or caused by LHRH receptor over-expression.

### RELATED APPLICATIONS

This application claims priority to U.S. Provisional Patent Application 62/961,107, filed Jan. 14, 2020, the contents of which are hereby incorporated by reference in its entirety.

### GOVERNMENT SUPPORT

This invention was made with government support under Award Number P20GM103436 awarded by the National Institute of Health. The government has certain rights in the invention.

### FIELD OF THE DISCLOSURE

The present disclosure relates to radiolabeled luteinizing hormone receptor hormone (LHRH) receptor binding compositions and methods of application and/or administration of the same for detecting cellular abnormalities, malignancies, tumors, and/or cancers.

### BACKGROUND

Breast and ovarian cancer are some of the most common cancers among individuals in the United States. Ovarian cancer is currently the seventh most commonly diagnosed cancer among women in the world with an estimated 239,000 new cases and 152,000 deaths worldwide. A women's lifetime risk is 1 in 75 for developing ovarian cancer and most are diagnosed at late stage where the five-year survival rate is only 29%. Despite advances for ovarian cancer detection there has been only a slight increase in survival rate (2-4%) since 1995 indicating the need for enhanced diagnostic and imaging mechanisms for earlier detection. See, e.g., B. M. Reid, et al., Cancer Biol. Med. 2017, 14 (1), (9-32). Similarly, breast cancer is currently the second leading cause of cancer death among women, accounting for approximately 570,000 deaths. The five-year survival rate is above 80%; however, for stage IV breast cancer the ten-year survival rate was found to be only 13%. See, e.g., Y. Sun, et al., Int. J. Biol. Sci. 2017, 13(11), (1387-1397) and L. G. Eng, et al., Breast Cancer Res. Treat. 2016, 160(1), (145-152).

Early and timely detection of cancer is noted to be the major factor dictating patient survival and prognosis. Mammogram screening has been critical for lowering mortality rates of breast cancer patients; however, it has limitations in women with dense fibrous tissue who may have false-negative and false-positive results. See, e.g., H. D. Nelson, et al., Ann. Intern. Med. 2016, 164(4), (226-35). Positron Emission Tomography (PET) imaging provides a more precise imaging of tumor position than x-ray or MRI through the use of injectable radioactive compounds; however, the accuracy is dependent on the radiopharmaceutical used. See S. Sofou, Int. J. Nanomedicine 2008, 3(2), (181-99)

Radionuclides such as both technetium (^{99m}Tc) and rhenium (^{186/188}Re) are transition metals that share common structural and chemical properties. See A. J. North, et al., Inorg. Chem. 2017, 56(16), (9725-9741). The decay characteristics of these radionuclides make them suitable for both cancer radioimaging and therapy. Moreover, in many studies non-radioactive Re analogues are used to module both ^{186/188}Re and ^{99m}Tc in a laboratory setting since radioactive properties are difficult to work with and the metals are similar in chemistry. See Garc, et al., Nucl. Sci. Tech. 2007, 18(2), (88-100).

^{99m}Tc is the most commonly used radionuclide in radiopharmaceutical imaging accounting for 70% of all procedures due to its small size and stability. See Garc, et al., Nucl. Sci. Tech. 2007, 18(2), (88-100). ^{99m}Tc emits detectable gamma rays (γ ray=142 keV) within a relatively short half-life of 6.02 hours with almost full decay at 24 hours, making ^{99m}Tc ideal for injection preparation with minimal radiation exposure to the patient. Consequently, ^{99m}Tc is used in many areas of radio imaging including brain imaging, bones scans, and vascular perfusion. See A. J. North, et al., Inorg. Chem. 2017, 56(16), (9725-9741); J. W. Babich, et al J. Nucl. Med. 1993, 34(12), (2176-81); M. D. Bartholoma, et al, Chem. Rev. 2010, 110(5), (2903-20).

Although ^{99m}Tc and ^{186/188}Re are currently used as radiotracers, each has limitations with respect to imaging sensitivity and specificity. The conjugation of peptide motifs with the radiometals would allow for earlier diagnosis, small metastatic colony imaging, and specific cancer cell targeting. For example, conjugation of peptide motifs to the radiotracers would provide rapid plasma clearance and high receptor targeting affinity. See J. W. Babich, et al., J. Nucl. Med. 1993, 34(12), (2176-81); A. J. Fischman, et al., J. Nucl. Med. 1993, 34(12), (2253-63).

The category of cancer cell type is characterized by the expression of certain receptors on the cancer cells surface. Receptors for specific hormones/peptides can be found to be overexpressed on the cancer cell compared to normal healthy cells. Reproductive cancers including breast, uterine, ovarian, prostate, testicular, etc., have been shown to overexpress the luteinizing hormone releasing hormone (LHRH) receptor. See M. Fekete, et al., J. Clin. Lab. Anal. 3 (3), pp. 137-47 (1989); A. Taheri, Int. J. Pharm. (2012). LHRH, also known as gonadotropin releasing hormone (GnRH), is a decapeptide with the sequence of pGlu-His-Trp-Ser-Tyr-Gly-Leu-Arg-Pro-Gly-NH₂ (or pEHWSYGLRPG-NH₂ SEQ ID NO: 1) (pGlu and pE being pyroglutamate or Pyr). LHRH is mainly involved in the regulation of reproduction. Additionally, various non-reproductive cancers overexpress the LHRH receptor as well, including lung, bladder, and pancreatic cancers, etc. See G. Carsten, et al., Front Endocrinol. 2017, (8), (187). The overexpression of the LHRH receptor on cancer cells makes LHRH an ideal candidate as a targeting peptide used in the field of radiopharmaceuticals. Further, rare and hard to treat cancers, such as triple negative breast and metastatic ovarian cancer, have been identified as overexpressing the LHRH receptor, making the application of the LHRH peptide to the field of radiopharmaceuticals an attractive endeavor to assist in earlier detection for significantly improved patient outcomes. See S. Seitz. et al., BMC Cancer 2014, (14), (847); G. Emons, et al., Hum. Reprod. 9 (7), pp. 1364-79, (1994).

Although the influence of LHRH on the administration of ^{99m}Tc was explored by Hao et al. (Oncol. Lett. 14: 569-578, 2017), that study involved mixing ^{99m}TcO₄⁻ directly with LHRH as two isolated compounds before injection. Mixing the separate compounds, however, creates only the possibility that a percentage of the formulation may display spontaneous combination of ^{99m}Tc and LHRH before application/injection. This combinatory method results in uncertainty regarding compound formation rendering it an ineffective and unreliable mode for imaging and further raising patient safety concerns. Other current solutions have attempted to introduce cysteine residues for cyclization of the peptide but these fail to ensure that the intrinsic properties of the LHRH peptide are maintained. See Barda et al., Nucl. Med. Biol. 31(7): 921-933 (2004).

Accordingly, there is a need for radiolabeled LHRH conjugates for detecting reproductive cancers that allow for the effective delivery of radiotracers, or radionuclides, such as ^{99m}Tc and ^{186/188}Re, to tumor cells.

### SUMMARY OF THE DISCLOSURE

In some aspects, the present disclosure concerns a luteinizing hormone releasing hormone (LHRH) composition of an LHRH peptide, a linker and a tridentate structure comprised of three amines and a metal nuclide, wherein the three amines retain the metal nuclide. In some aspects, the linker is covalently bound internally to the LHRH peptide.

In some aspects, the present disclosure concerns an LHRH composition, wherein the LHRH peptide includes an amino acid sequence as set forth in SEQ ID NO: 2 or SEQ ID NO: 3, wherein X1 is a serine residue, X2 is a tyrosine, phenylalanine, leucine or histidine residue, X3 is glycine, leucine, serine, histidine, lysine, aspartate, glutamate, alanine, or tryptophan residue and X4 is a leucine, valine, tryptophan, or methionine residue. In some aspects, the linker is covalently bound to X1, X2, X3 or X4. In further aspects, one or more of X1, X2, X3 or X4 is a right-handed amino acid.

In some aspects, the present disclosure concerns LHRH compositions an LHRH peptide, a linker and a tridentate structure comprised of three amines and a metal nuclide where the LHRH peptide is selected from the group consisting of [D-Lys6]-LHRH, leuprorelin, goserelin, buserelin, histrelin, triptorelin, degrelix, nafarelin, [D-Trp6]-LHRH, [D-Ala6]-LHRH, [Gln8]-LHRH, antide, and gonadorelin. In some aspects, the LHRH peptide is [D-Lys6]-LHRH.

In other aspects, the present disclosure concerns LHRH compositions an LHRH peptide, a linker and a tridentate structure comprised of three amines and a metal nuclide wherein the linker is covalently bound to the LHRH peptide through a phenyl, an amine, a hydroxyl, a sulfhydryl, a thiol, or a carboxyl group on a side chain of an amino acid therein. In some aspects, the linker is covalently bound to an amine of a side chain of the LHRH peptide. In further aspects, the linker includes an amino alkyl carboxylic acid, wherein the alkyl is of between 1 to 25 carbons in length. In further aspects, the linker may further include a polyethylene glycol chain of between 1 to 50 repeats in length.

In some aspects, the present disclosure concerns LHRH compositions of an LHRH peptide, a linker and a tridentate structure comprised of three amines and a metal nuclide wherein the tridentate structure is covalently linked to the linker. In some aspects, the tridentate structure is a dialkyltriamine. In further aspects, the tridentate structure is a diethylenetriamine.

In some aspects, the present disclosure concerns LHRH compositions of an LHRH peptide, a linker and a tridentate structure comprised of three amines and a metal nuclide wherein the metal nuclide is a radionuclide. In some aspects, the radionuclide can be selected from ^{99m}technetium, ¹⁸⁶rhenium, ¹⁸⁸rhenium, ^{186/188}rhenium, ⁶⁷gallium, ⁶⁸gallium, ⁶⁴copper, ⁶⁷copper, ^{64/67}copper, ⁹⁰yttrium, ¹⁷⁷lutetium, ¹⁹²iridium, ¹⁰³palladium, ⁸⁹strontium, ¹⁵³samarium, ²¹²lead, ²¹³bismuth, ¹³¹caesium, ²²³radium, ²²⁵radium, ²²⁵actinium, ²²⁸thorium, ²²⁷thorium, ²¹¹astatine, and ¹¹¹indium. In further aspects, the radionuclide is ^{99m}technetium. In other aspects, the radionuclide is ^{186/188}rhenium. In some aspects, the present disclosure concerns needed LHRH compositions to provide precise tumor size and placement imaging, earlier and smaller colony formation detection, and specific cancer cell targeting leading to better patient treatments and outcomes.

In some aspects, the present disclosure concerns LHRH compositions of an LHRH peptide, a linker and a tridentate structure comprised of three amines and a metal nuclide wherein the LHRH peptide is dimerized through the metal nuclide.

In some aspects, the present disclosure concerns LHRH compositions of an LHRH peptide, a linker and a tridentate structure comprised of three amines and a metal nuclide a pharmaceutically acceptable carrier.

In other aspects, the present disclosure concerns methods for labeling an LHRH receptor on a cell comprising administering an LHRH composition of an LHRH peptide, a linker and a tridentate structure comprised of three amines and a metal nuclide, whereby the LHRH composition binds the LHRH receptor to provide a label thereof. In some aspects, the cell is in vitro, including cells from a biopsy or a tissue sample. In other aspects, the cell is in vivo.

In some aspects, the present disclosure concerns methods for determining LHRH receptor expression in a subject by administering the LHRH composition as set forth herein and detecting the metal nuclide thereof. In some aspects, the methods include administering the LHRH compositions to a subject.

The present disclosure further concerns, in some aspects, an LHRH composition include a structure as set forth in formula I:

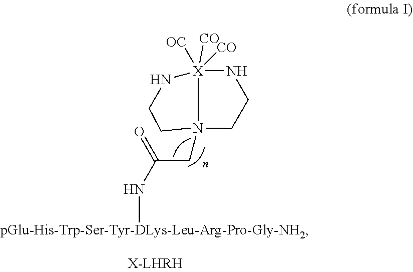

wherein X is a radionuclide and n is from 1 to 25.

In other aspects, the present disclosure concerns an LHRH composition that include a structure as set forth in formula II:

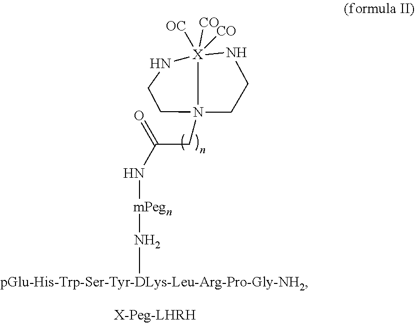

wherein X is a radionuclide and n is from 1 to 25 and mPegₙ is from 1 to 50.

In some aspects, for either formula I or formula II, X can be selected from rhenium, ^{99m}technetium, ¹⁸⁶rhenium, ¹⁸⁸rhenium, ^{186/188}rhenium, ⁶⁷gallium, ⁶⁸gallium, ⁶⁴copper, ⁶⁷copper, ^{64/67}copper, ⁹⁰yttrium, ¹⁷⁷lutetium, ¹⁹²iridium, ¹⁰³palladium, ⁸⁹strontium, ¹⁵³samarium, ²¹²lead, ²¹³bismuth, ¹³¹caesium, ²²³radium, ²²⁵radium, ²²⁵actinium, ²²⁸thorium, ²²⁷thorium, ²¹¹astatine, and ¹¹¹indium.

In further aspects, the present disclosure concerns LHRH compositions that include a structure as set forth in Formula III or IV:

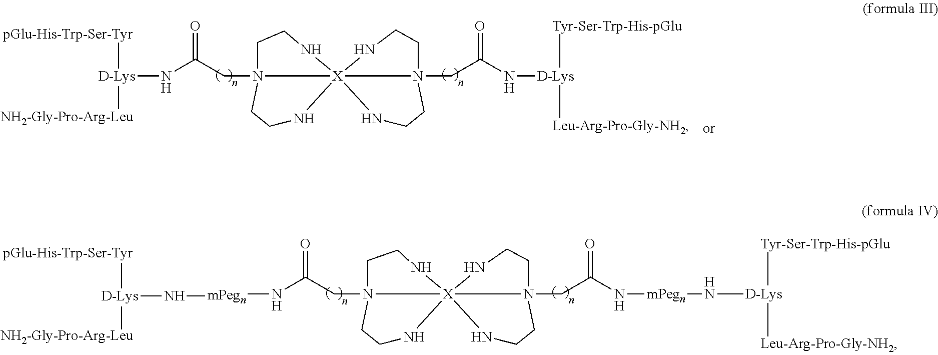

wherein X is a radionuclide of a transition or post-transition metal and n is from 1 to 25 and mPegₙ is from 1 to 50. In further aspects, X can be selected from ^{99m}Tc, ^{186/188}Re, ¹¹¹ln, ⁶⁸Ga, ^{64/67}Cu, ⁹⁰Y, and ¹⁷⁷Lu.

### BRIEF DESCRIPTION OF THE DRAWINGS

FIG. 1A depicts synthesis of ^{99m}Tc/Re-Acdien-LHRH. The synthesis of an Acdien-LHRH composition was performed using solid phase chemistry with subsequent conjugation with Re and Tc analogues (with or without a miniPEG group in the linker).

FIG. 1B depicts an exemplary synthesis of a dialkyltriamine linker in accordance with a preferred embodiment of the present disclosure;

FIG. 2A depicts an exemplary synthesis of a radionuclide linked to an LHRH peptide in a dimer formation with the two LHRH peptides linked through a shared nuclide or radionuclide. FIG. 2B depicts the same as 2A with the inclusion of the PEG intermediary in the linker.

FIG. 3 depicts a mass spectrum of Rhenium-Acdien-LHRH-LHRH dimer composition in accordance with one aspect of the present disclosure. FIG. 3 shows a mass spectrum of Re—LHRH conjugate—ESI-MS (TOF) (M+H)⁺ calculated for C₆₉H₁₀₂N₂₁O₁₇Re 1684.73; found 1684.7023.The most intense peak (base peak) at 562.24 and 842.8590 are the triply charged and doubly charged peaks respectively.

FIG. 4 depicts a mass spectrum of Re-Acdien-PEG-LHRH conjugate in accordance with one aspect of the present disclosure. FIG. 4 shows a mass spectrum of Re-Acdien-PEG-LHRH conjugate—ESI-MS (TOF) (M+H)⁺ calculated for C₇₆H₁₁₂N₂₂O₂₂Re 1875.07; found the most intense peak (base peak) at 625.2813 and 937.4119. These are the triply charged and doubly charged peaks respectively.

FIG. 5 depicts results of cell viability following administration of both Rhenium and a Rhenium-Acdien-LHRH composition against 4T1 breast cancer cells in a MTT proliferation assay, along with, a cellular uptake assay in accordance with one aspect of the present disclosure. FIG. 5A shows MDA-MB-231 cells treated with Re or Re-Acdien-LHRH from 0.1-100 uM for 24 hrs. Viabilty rates were analyzed by a MTT assay after 48 hrs incubation. FIG. 5B shows MDA-MB-231 cells treated with 100 uM of Re or Re-Acdien-LHRH for 24 hrs. Cells were collected and Re concentration mg/L was measured using ICP-MS. n=3, two-way Anova and unparied t-test; ****p<0.0001.

FIG. 6 depicts cytotoxicity results after treatment with Rhenium and a Rhenium-Acdien-LHRH composition (w/PEG included in the linker) against 4T1 and MDA-MB-231 breast cancer cells in a MTT proliferation assay, along with, cellular uptake assays in accordance with some aspects the present disclosure. FIG. 6A shows MDA-MB-231 and FIG. 6B shows 4T1 cells treated with Re or Re-Acdien-LHRH from 0.1-100 uM for 24 hrs. Viabilty rates for both were analyzed by a MTT assay after 48 hrs incubation. FIG. 6C shows MDA-MB-231 and FIG. 6D shows 4T1 cells treated with 100 uM of Re or Re-Acdien-LHRH for 24 hrs. Cells were collected and Re concentration mg/L was measured using ICP-MS. n=3, two-way Anova and unparied t-test; ****p<0.0001.

### DETAILED DESCRIPTION

The present disclosure provides exemplary compositions and applications of such for the present disclosure, reference is made to the accompanying drawings that form a part hereof, and show by way of illustration specific manners in which the embodiments may be practiced. It is to be understood that other embodiments may be utilized and changes may be made without departing from the understood scope.

The present disclosure concerns luteinizing hormone releasing hormone (LHRH) compositions of LHRH peptides and derivatives thereof. As set forth herein, an LHRH peptide is based on a ten amino acid peptide with a sequence as set forth in SEQ ID NO: 1 (Pyr-His-Trp-Ser-Tyr-Gly-Leu-Arg-Pro-Gly-NH₂) (Pyr being pyroglutamate). In other aspects, an LHRH peptide may be missing the amino terminal glycine. In some aspects, the LHRH peptides are conjugated to another domain(s) to form an LHRH composition. A composition that is a “conjugate” of two domains refers to one in which the two domains (or moieties) are covalently bonded to one another, either directly or via a linker.

In some aspects, the LHRH compositions can include LHRH peptides that include the three most carboxyl terminal and three most amino terminal amino acids as set forth in SEQ ID NO: 1, with between 2 and 10 amino acids spaced therebetween, including 3, 4, 5, 6, 7, 8, and/or 9. In other aspects, an LHRH peptide may include a peptide or modified form thereof that retains ability to bind with an LHRH receptor. Such peptides may bind the LHRH receptor with similar, reduced or enhanced affinity.

In some aspects, LHRH peptides can include peptides with the amino acid sequence Pyr-His-Trp-X1-X2-X3-X4-Arg-Pro-Gly-NH₂ (SEQ ID NO: 2) and/or Glu-His-Trp-X1-X2-X3-X4-Arg-Pro-Gly-NH₂ (SEQ ID NO: 20) and/or Pyr-His-Trp-X1-X2-X3-X4-Arg-Pro-NH₂ (SEQ ID NO: 3) and/or Glu-His-Trp-X1-X2-X3-X4-Arg-Pro-NH₂ (SEQ ID NO: 21), wherein X1, X2, X3 and X4 are any amino acid. In some aspects, X1 is a serine residue and/or X2 is a tyrosine, phenylalanine, leucine or histidine residue and/or X3 is glycine, leucine, serine, histidine, lysine, aspartate, glutamate, alanine, or tryptophan residue and/or X4 is a leucine, valine, tryptophan, or methionine residue. In further aspects, X1 and/or X2 and/or X3 and/or X4 is a right-handed or “D” isomer residue, such a D-Ala, D-His, D-Lys, D-Leu, D-Ser, D-Met, D-Val, D-Tyr, D-Asp, D-Glu or D-Trp residue. It will be appreciated that the LHRH peptides may include the presence of one or more of a β-amino acid, a γ-amino acid, a proline derivative, 2-naphthyalanine, biphenylalanine, N-alkyl glycine, peptoids, a 3-substituted alanine, a glycine derivative, a ring-substituted phenylalanine, a ring-substituted tyrosine, and/or an N-methyl amino acid. In some aspects, X1 and/or X2 and/or X3 and/or X4 is appended with a further modification, such as a napthyl, t-butyl (tBu), benzyl, isopropyl, ureido, S-dihydroorotamido, and/or pyridyl group.

In some aspects, LHRH peptides may optionally be devoid of an amino terminal glycine. In other aspects, the LHRH peptides may feature an ethylamide (NHEt), hydrazide, and/or a carbomyl group at one or both termini.

In some aspects, the LHRH peptides can include various isoforms or analogs of LHRH with a radiolabel. An isoform or analog may refer to a molecule of a peptide sequence that is comparable to an endogenously produced compound, in which, one or more residues have been replaced, deleted or modified. An isoform or analog of LHRH can include natural or synthetic peptides that resemble endogenous LHRH in structure and/or function. Such may include the peptides pEHWSHGWYPG-NH₂ (SEQ ID NO: 4), EHWSHGWYPG-NH₂ (SED ID NO: 22), pEHWSHDWKPG-NH₂ (SEQ ID NO: 5), and EHWSHDWKPG-NH₂ (SEQ ID NO: 23). In further aspects, LHRH peptides can include analogous peptides with varying chemical modification(s) and/or appended groups to the peptides therein. For example, currently identified analogous peptides include: [D-Lys6]-LHRH (Pyr-His-Trp-Ser-Tyr-D-Lys-Leu-Arg-Pro-Gly-NH₂)(SEQ ID NO: 6) or (Pyr-His-Trp-Ser-Tyr-D-Lys-Leu-Arg-Pro-NH₂) (SEQ ID NO: 7), leuprorelin (Pyr-His-Trp-Ser-Tyr-D-Leu-Leu-Arg-Pro-NHEt) (SEQ ID NO: 8), goserelin (Pyr-His-Trp-Ser-Tyr-D-Ser(tBu)-Leu-Arg-Pro-NHNHCONH₂) (SEQ ID NO: 9), buserelin (Pyr-His-Trp-Ser-Tyr-D-Ser(tButyl)-Leu-Arg-Pro-NHEt) (SEQ ID NO: 10), histrelin (Pyr-His-Trp-Ser-Tyr-D-His(1-Benzyl)-Leu-Arg-Pro-NHEt) (SEQ ID NO: 11), triptorelin (Pyr-His-Trp-Ser-Tyr-D-Trp-Leu-Arg-Pro-Gly-NH₂) (SEQ ID NO: 12), degrelix (Acetyl-D-Ala(2-Naphthyl)-D-Phe(4-Chloro)-D-Ala(3-Pyridyl)-Ser-Phe(4-S-dihydroorotamido)-D-Phe(4-ureido)-Leu-Lys(isopropyl)-Pro-D-Ala-NH₂) (SEQ ID NO: 13), nafarelin (Pyr-His-Trp-Ser-Tyr-D-Ala(2-naphthyl)-Leu-Arg-Pro-Gly-NH₂) (SEQ ID NO: 14), [D-Trp6]-LHRH (Pyr-His-Trp-Ser-Tyr-D-Trp-Leu-Arg-Pro-Gly-NH₂) (SEQ ID NO: 15), [D-Ala6]-LHRH (Pyr-His-Trp-Ser-Tyr-D-Ala-Leu-Arg-Pro-Gly-NH₂) (SEQ ID NO: 16), [Gln8]-LHRH (Pyr-His-Trp-Ser-Tyr-Gly-Leu-Gln-Pro-Gly-NH₂) (SEQ ID NO: 17), antide (Acetyl-D-2Nal-D-Phe(4-Cl)-D-3Pal-Ser-Lys(nicotinyl)-D-Lys(nicotinyl)-Leu-Lys(isopropyl)-Pro-D-Ala-NH₂) (SEQ ID NO: 18), and gonadorelin (Pyr-His-Trp-Ser-Tyr-Gly-Leu-Arg-Pro-Gly-NH₂) (SEQ ID NO: 19). It will be apparent to those skilled in the art that each instance of pyroGlu can be readily replaced with glutamate. In addition, LHRH peptides may include LHRH receptor agonists and antagonists. See A. V. Schally, et al., Prostate, 45, pp. 158-66 (2000); W. R. Miller, Nature, 313, pp. 231-33 (1985); and K. Szepashzi, et al., Breast Cancer Res. Treat., 56, pp. 267-76 (1999).

In some aspects, the LHRH peptides of the LHRH compositions can be recombinant or synthetic peptides or a combination thereof. Recombinant peptides can be generated and/or isolated from cells that include a nucleic acid sequence encoding the LHRH peptides, such as through the introduction of a DNA or RNA molecule with a nucleic acid encoding the LHRH peptide to a cell, such as through transfection or transformation. Recombinant LHRH peptides may further be prepared using cell-free protein synthesis (CFPS) or in vitro protein synthesis with a cellular lysate including ribosomes, t-RNA synthetases, nucleases and elongation factors and the like to translate a nucleic acid encoding the LHRH peptide without the confines of a cell wall.

In other aspects, the LHRH peptides can be assembled synthetically. Such may include solution phase synthesis and/or solid phase synthesis and may include a stepwise assembly of the LHRH peptide's amino acids through application of amino acids with an amino terminal protecting group and/or a side chain protecting group that can be selectively de-protected to allow for the appropriate formation of the peptide bonds as each amino acid is added.

In some aspects, the present disclosure also concerns post-translational or post-synthesis LHRH peptide modifications. Such can include natural modifications that occur or can occur in a cell, and non-natural chemical modifications. By way of example and not limitation, such can include phosphorylation, glycosylation, methylation, acetylation, isoprenylation, myristolation, palmitoylation, alkylation, amidation, butyrylation, hydroxylation, iodination, adenylation, succinylation, sulfation, glycation, carbamylation, carbonylation, biotinylation, oxidation, and/or PEGylation (polyethylene glycol) of one or more amino acids.

In some aspects, the LHRH peptides may be appended and/or conjugated with a linker. A linker may include an amino, a carboxylic, an ester, an ether, or an aliphatic alkyl covalent attachment to a side chain of one or more amino acids of the LHRH peptides as set forth herein. A linker may also include acetate linkers, PEG groups or chains thereof, ester linkers, sugars, lectins, antibodies and their fragments, hormones and hormone analogues. In some aspects, a further LHRH peptide may serve as a linker.

In some aspects, the linker may covalently bind to a side chain of an amino acid of the LHRH peptide, such as through a reactive aspect of a side chain, including a phenyl, an amine, a hydroxyl, a sulfhydryl, a thiol, and/or a carboxyl group. In some aspects, a linker may include the covalent attachment of an amino aliphatic carboxylic acid, such as an amino alkyl carboxylic acid, including saturated or non-saturated and/or hydroxylated alkyl chains of between 1 and 25 carbons in length (e.g. —(CH₂)ₙ— wherein n=1-25), including 2, 3, 4, 5, 6, 7, 8, 9, 10, 11, 12, 13, 14, 15, 16, 17, 18, 19, 20, 21, 22, 23, and 24 carbons in length. In further aspects, the terminal carbon of an appended amino aliphatic carboxylic acid may be linked to the central nitrogen of a di-amino di-alkyl amine or a dialkyltriamine, such as diethylenetriamine or dien for short.

In some aspects, a linker may also include a polyethylene glycol (PEG) attached and/or conjugated to a reactive component of a side chain of an amino acid residue of the LHRH peptides as set forth herein. In some aspects, the PEG group or chain may attach to an available amine on a side chain. In some aspects, the PEG group or chain may be of between 1 to about 50 ethylene oxide repeats, including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49 ethylene oxide repeats. In further aspects, the terminal hydroxyl of the PEG may be linked to an amino aliphatic carboxylic acid, such as an amino alkyl carboxylic acid.

In further aspects, other linkers may be present and/or included to connect the LHRH peptide with the amino aliphatic carboxylic acid linker. Such may include, either in addition to PEG groups or chains or as a substitution for PEG groups or chains, sulfonamide, oxo, triazine and/or pyridyl ligands. Similarly, additional peptide or hormones may also be used as linkers or as a part thereof.

In some aspects, the LHRH peptide is linked to a di-aminoalkyl amine or a dialkyltriamine, such that three amine groups are present to allow for provide for a tridentate structure of the LHRH composition. In some aspects, the tridentate structure is conjugated to the linker that is conjugated to the LHRH peptide. In some aspects, the tridentate structure can bind and/or retain a transition/post-transition metal or an isotope or a radioisotope thereof.

The present disclosure, in some aspects, provides for compositions of LHRH peptides linked to a tridentate structure bearing a metal, such as an alkali, alkaline earth, transition or post-transition metal nuclide, or halide. Such elements are understood in the art. In some aspects, the alkali/alkali earth/transition/post-transition metal or halide nuclide is an isotope thereof, including a radio-isotope or radionuclide such that it is a radiation emitter, such as an alpha, beta or gamma ray emitter. By coupling to LHRH peptides, the radioactive alkali/alkaline earth/transition/post-transition metal or halide nuclide can be delivered with specificity to LHRH receptors and/or cells expressing LHRH receptors. Through delivery of a radionuclide-label to LHRH receptors, the compositions provide for radiolabeling of cells over-expressing LHRH receptors and/or the identification of cells over-expressing LHRH receptors. In instances where cells overexpress LHRH receptors, the LHRH-radioactive metal compositions can selectively deliver radiolabels and/or radioactive treatment.

In some aspects, the tridentate structure retains and/or binds a radioactive alkali, alkaline-earth, transition or post-transition metal or halide nuclide. Radionuclide can also be referred to as radiolabels. Examples of suitable radio-isotopes or radionuclides include ¹⁸fluorine, ^{99m}technetium, ¹⁸⁶rhenium, ¹⁸⁸rhenium, ^{186/188}rhenium, ⁶⁷gallium, ⁶⁸gallium, ⁶⁴copper, ⁶⁷copper, 64/67copper, ⁹⁰yttrium, ¹⁷⁷lutetium, ¹⁹²iridium, ¹⁰³palladium, ⁸⁹strontium, ¹⁵³samarium, ²¹²lead, ²¹³bismuth, ¹³¹caesium, ²²³radium, ²²⁵radium, ²²⁵actinium, ²²⁸thorium, ²²⁷thorium, ²¹¹astatine, and ¹¹¹indium.

The present disclosure accordingly provides for LHRH compositions of LHRH peptides coupled or conjugated, directly or indirectly through a linker, to an alkali, alkaline earth, transition or post-transition metal or halide nuclide or isotope thereof. Such compositions may utilize and amino aliphatic carboxylic acid and/or a PEG linker and/or other linker as an intermediary as set forth herein. The LHRH peptide may be coupled or conjugated to a di-aminoalkyl amine or a dialkyltriamine to provide a tridentate structure to retain the metal or halide nuclide or isotope thereof.

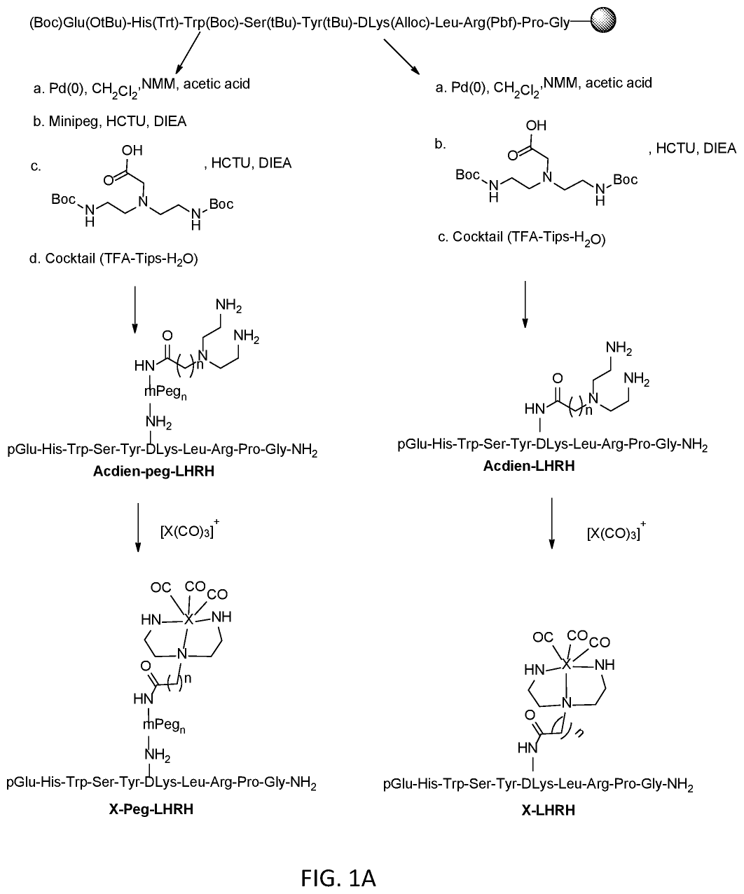

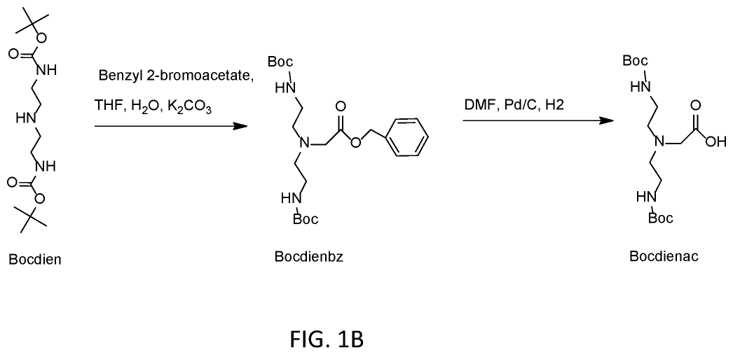

In some aspects, the present description concerns LHRH-compositions that feature a peptide fragment, a linker fragment and a tridentate fragment, the latter of which can retain a metal or halide nuclide or a radionuclide thereof. FIGS. 1A and 1B demonstrate two aspects of the LHRH compositions as set forth herein with a schematic outlining the synthesis thereof In some aspects, the LHRH composition includes a structure as set forth in Formula I:

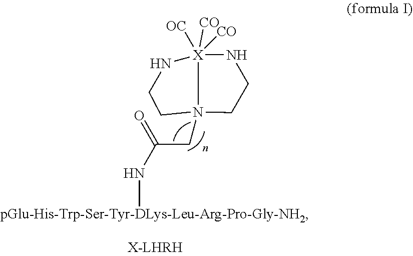

wherein X is a radionuclide and wherein n is from 1 to 25. In other aspects, the LHRH composition includes a structure as set forth in Formula II:

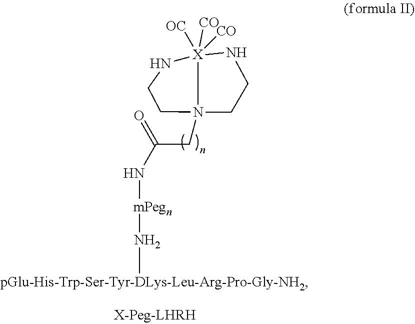

wherein X is a radionuclide and wherein n is from 1 to 25 and mPEGₙ is of between 1 and 50. The peptides demonstrated in Formulas I and II utilizes a D-Lysine residue in the internal segment of the LHRH peptide, leaving the N- and C-terminal amino acids required for LHRH receptor binding to ensure that the intrinsic receptor binding properties of the LHRH peptide are unaltered by any further modification and/or conjugation. A di-amino ethyl amine can then be reacted with or without a PEG intermediary (cf. Formulas I and II) to provide one version of the LHRH compositions as set forth herein. FIG. 1B shows an overview of one approach to prepare a di-amino alkyl amine with the use or presence of protecting groups on the primary amines to allow the secondary internal amine to link to the LHRH peptide.

FIG. 1A further sets forth a schematic depicting the introduction and the retention of a metal by the tridentate structure present in the LHRH compositions set forth herein. As set forth in the FIG. 1A, once the di-aminoalkyl amine or dialkyltriamine is linked to the LHRH peptide, the protecting groups can be removed and when reacted with an acid cocktail to complete the LHRH composition. FIG. 1A demonstrates the success of the tridentate retention where the depicted LHRH peptide is conjugated to ^{99m}Tc and ^{186/188}Re using the Bocdienac linker as described herein on a right-handed lysine substituted for glycine at position 6 of SEQ ID NO: 1 (NH₂ depicted at the peptide is from the lysine residue).

As also set forth in FIG. 1A, an LHRH peptide may be conjugated to a nuclide both with and without the additional presence of Polyethylene glycol (PEG) groups or chains as the LHRH peptide conjugates (radiolabeled or not) do not require a PEGylated linker or similar to be soluble and effective. As noted herein, however, PEGylation can further provide enhanced solubility or bioavailability of synthetic compounds, as seen with some nanoparticles and dendrimers. See, e.g., J. Heldt, et al., J. Organomet. Chem, 689, (2004) 4775-4782; N. Nukolova, Mol. Pharmaceutical, 10 (10), pp. 3913-21 (2013); and U.S. Published Patent Application No. 2011/0104074 A1.

Accordingly, in some aspects of the disclosure, an LHRH peptide can be conjugated to a radionuclide, such as ^{99m}Tc or ^{186/188}Re as shown in FIG. 1A, using a linker, such as the depicted Bocdienac linker and optional Polyethylene glycol (PEG) groups or chains. As is also described herein, other linkers, including, but not limited to, additional ligands (sulfonamide, oxo, triazine and pyridyl ligands), peptides, or hormones (such as LHRH and its analogs), may also be used.

FIG. 1A sets forth an exemplary approach for conjugating rhenium to an LHRH peptide. In synthesizing a ^{186/188}Re-LHRH conjugate, [^{186/188}Re(CO)₃(H₂O)₃]Br is used to bind to LHRH couple to an acetyl diethylenetriamine (Acdien), both with and without an intermediary PEG. The LHRH moiety of provides an effective approach to directly provide the radionuclide to cells expressing LHRH receptors. In comparison to administering a radionuclide alone, such as [Re(CO)₃(H₂O)₃]Br, the LHRH compositions provide effective systemic distribution and bioavailability. In addition, herein discovered, that due to the increase in cellular internalization of Re-LHRH compared to Re alone provides an indication of no release or cleavage of the Re(CO₃) moiety from the peptide upon 24 hour solubilization demonstrating stability of the complex.

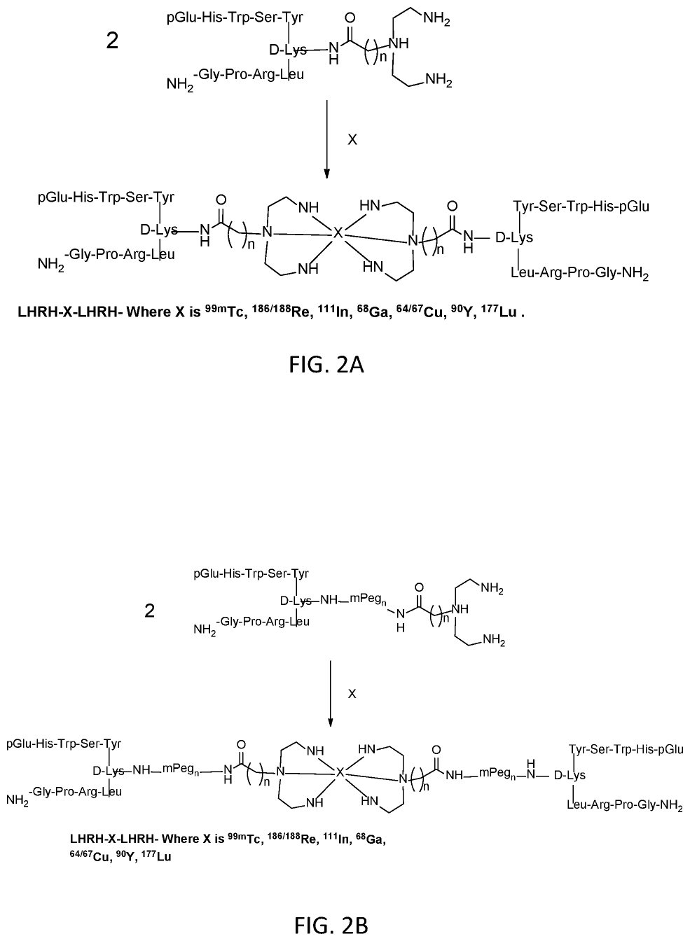

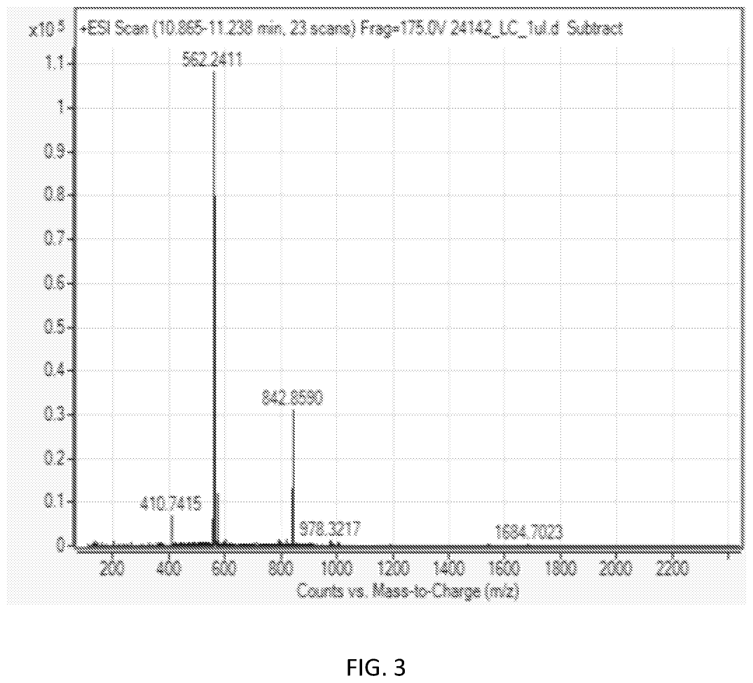

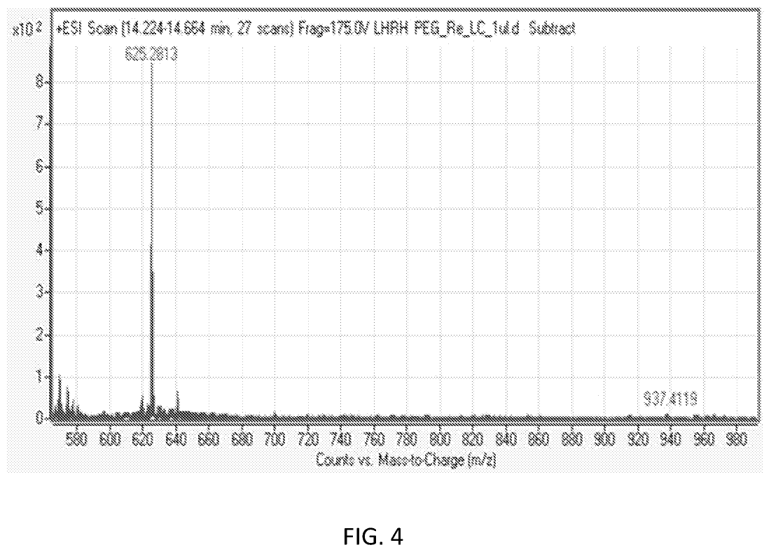

FIG. 1B shows an example for synthesizing a tridentate portion of the LHRH compositions as set forth herein. As set for in FIG. 1B, a di-aminoalkyl amine or aialkyltriamine such as diethylenetriamine can be prepared with protecting groups such as Boc in FIG. 1B to leave the central amine open for further conjugation. As depicted, the central amine can then conjugate to the terminal carbon of an alkyl carboxylic acid. As shown in FIG. 2, the carboxyl group can then provide for in alternative embodiments ^{186/188}Re may be substituted with any other radionuclides, including but not limited to ^{99m}Tc, ¹¹¹ln, ⁶⁸Ga, ^{64/67}Cu, ⁹⁰Y, and ¹⁷⁷Lu, and any derivatives thereof.

In further aspects, the LHRH compositions may include a dimer and/or be formulated as a dimer. As set forth in the working examples and in, e.g., FIG. 1A, a transition/post-transition metal nuclide or radio-isotope thereof retains about three carbonates when encapsulated by the tridentate structure of the LHRH compositions. Accordingly, such may be displaced by the introduction of a second LHRH peptide composition to allow for the formation of a dimer that shares or is linked by the metal nuclide (see, e.g., FIGS. 2A and B) or an LHRH-X-LHRH composition, wherein X is a transition or post-transition metal nuclide or radio-isotope thereof.

FIG. 2 sets forth a further aspect of the disclosure, wherein LHRH peptides can dimerize through 2 of the LHRH compositions sharing a metal nuclide. Formula III presents an exemplary example of such:

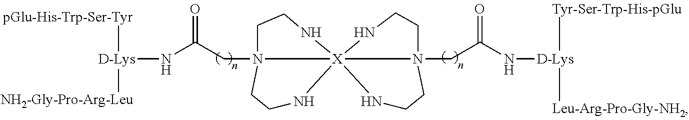

wherein X is a metal nuclide and n is of between 1 and 25. As set forth, the LHRH-linker-tridentate structures can arrange either side of a shared metal nuclide, X, providing a dimer LHRH-X-LHRH. It will be appreciated that as depicted in Formula III, the presence of PEG in such dimers is a further aspect of the disclosure, as is depicted in Formula IV:

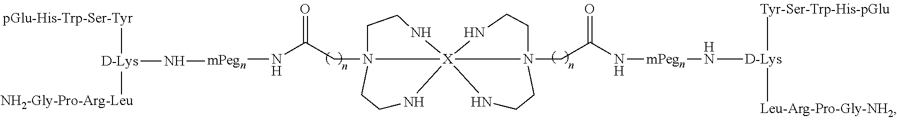

wherein X is a metal nuclide and n is of between 1 and 25 and mPEGₙ is of between 1 and 50.

In some aspects, the present disclosure concerns methods for preparing the LHRH compositions as set forth herein. In some aspects, the method can utilize synthetic approaches for assembling the LHRH compositions, such as through the use of solution phase synthesis or solid-phase synthesis (see, e.g., Isidro-Llobet et al. J. Org. Chem. 84(8): 4615-4628, 2019; Stawikowski et al., Curr. Protoc. Protein. Sci. 26(1): 18.1.1-18.1.9, 2001)). As set forth in the working examples, LHRH peptides can be assembled using solid state protein synthesis with Fmoc (9-fluorenyl methoxy carbonyl) techniques. The protection of the desired side chain with tert-butyloxy carbonyl (Boc) allows for the peptide to be assembled and then the addition of the linker and the tridentate structure. As set forth in the examples, Boc groups may also be utilized to protect the amino ends of the tridentate structure and allow for the free amine of the amino aliphatic carboxylic acid to bind and/or couple to the LHRH peptide or PEG group appended thereto. For convenience, the assembled linker and the tridentate structure with the optional presence of one or more PEG groups is referred to in some parts of the disclosure as a Bocdienac (Boc-dien-acetyl) linker. It will be appreciated that the terminology does not require the presence of a Boc group in the final composition, but may instead refer to the application of Boc to protect the amines of tridentate structure during the preparation of the LHRH compositions.

In some aspects, the present disclosure provides radionuclide LHRH compositions. As set forth herein, the tridentate structure can retain an isotope of an alkali, alkaline earth, transition or post-transition metal or halide, such as a radionuclide. In some aspects, the LHRH composition is a dimer around a shared radionuclide. In some aspects, the present disclosure provides ^{99m}Tc, ¹⁸⁶Re, ¹⁸⁸Re, ^{186/188}Re, ¹⁸F, ⁶⁷Ga, ⁶⁸Ga, ⁶⁴Cu, ⁶⁷Cu, ^{64/67}Cu, ⁹⁰Y, ¹⁷⁷Lu, ¹⁹²Ir, ¹⁰³Pd, ⁸⁹Sr, ¹⁵³Sm, ²¹²Pb, ²¹³Bi, ¹³¹Cs, ²²³Ra, ²²⁵Ra, ²²⁵Ac, ²²⁸Th, ²²⁷Th, ²¹¹At and ¹¹¹In-LHRH peptide dimers or conjugates.

The radionuclide-LHRH compositions, through the binding to LHRH receptors, allow for an effective and site-specific delivery of the respective radionuclides to enhance diagnosis and/or observation and/or monitoring of tumor cells. The specific targeting of the LHRH receptor by the LHRH compositions as set forth herein increases the precision, sensitivity and accuracy of imaging by accumulating a higher concentration of the radionuclides at the tumor cells overexpressing the LHRH receptors, thereby decreasing background and/or non-specific radiolabeling.

In other aspects, the present disclosure concerns methods of administering the LHRH compositions as set forth herein. Such administration may be providing the LHRH compositions to a cell, such as a cell in vitro, in vivo, or ex vivo. Such methods may include administering the LHRH compositions as set forth herein to the extra-cellular space. By administering the LHRH compositions to an extra-cellular area, the compositions may come into contact and/or proximity with the cell-surface expressed LHRH receptors. Due to the retained affinity for the LHRH receptor by the LHRH compositions, the LHRH composition can be retained or coupled to the receptor on the cell-surface. In some aspects, binding to the LHRH receptor may further provide for intracellular labeling through the internalization of the LHRH receptor after the LHRH composition binds thereto.

The LHRH compositions may be administered as a solution. As set forth herein, the LHRH compositions retain good solubility, although solubility may be enhanced if needed through the additional presence of PEG groups or chains in the linker. The LHRH compositions may be administered as a solution to a cell media solution or systemically to a subject. Routes of administration are well understood in the art and may include intravenous injection, oral ingestion, intrathecal injection, transdermal application, ocular application, nasal application, otic application, rectal administration and/or mucous membrane application. Administration may be systemic or local, such as to a tissue suspected or known to over express LHRH receptors. It will also be appreciated that the LHRH compositions may be administered to cells for a pathological analysis, such as in examining a biopsy or tissue obtained from a subject. A “subject” as set forth herein may refer to any animal or cell derived therefrom. In some aspects, a subject may include a mammal, such as a human. In some aspects, a subject refers to a human subject.

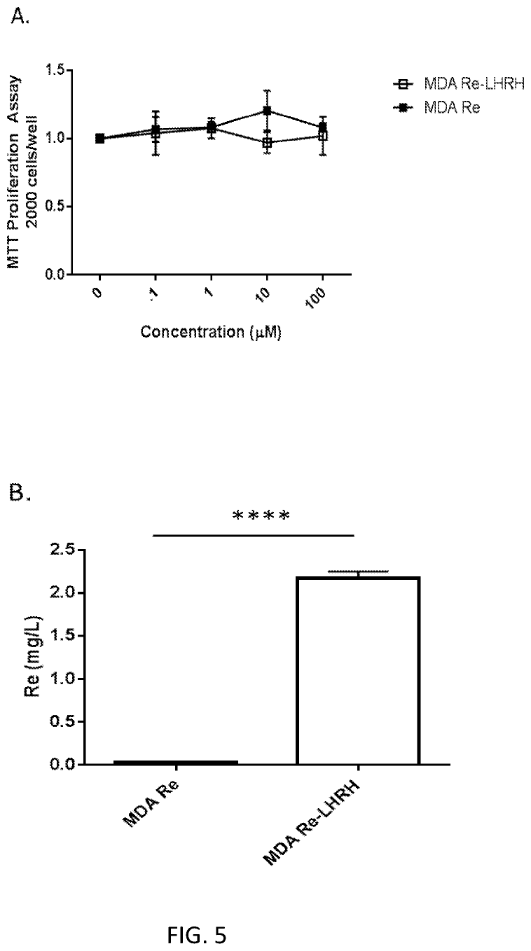

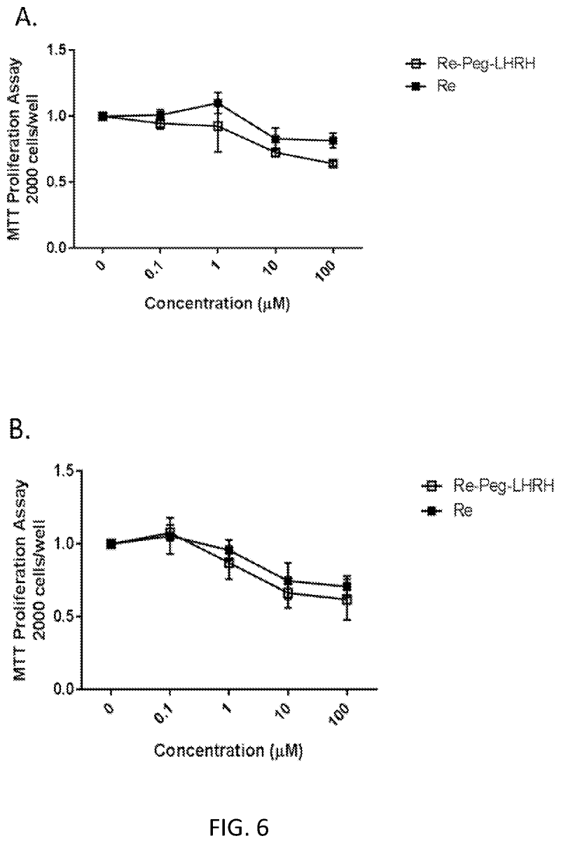

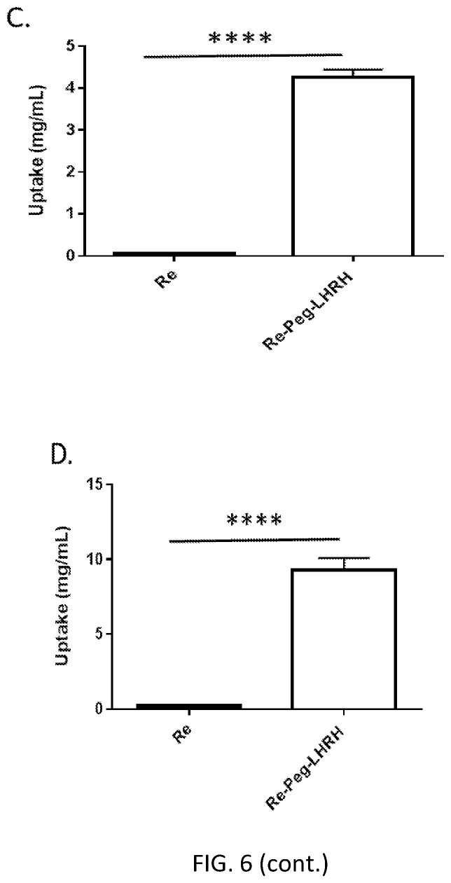

In some aspects, the LHRH-compositions may be administered, such as to a cell or a subject, and then detected to image and/or identify the location and/or concentration of LHRH receptors in a subject or cellular sample. As set forth herein, the LHRH compositions may include radionuclides, which may in turn be detected by means understood in the art, such as positron emission tomography, gamma cameras, single photon emission tomography, and the like. As set forth herein, LHRH receptors can be over-expressed in certain types of abnormally growing cells, including those associated with tumorous or malignant or cancerous growth. Accordingly, after administering the LHRH-compositions as set forth herein, in aspects where a radionuclide is part of the LHRH-composition, through detecting and/or imaging the radionuclides, due to binding the LHRH receptor, the LHRH compositions allow for cellular internalization after binding the LHRH compositions and selective labeling of such cells with a radionuclide. As described in the working examples, in administering the compositions as set forth herein to cells from breast cancer cell lines, both a Rhenium-LHRH and Rhenium-PEG-LHRH did not result in any alteration in normal cell growth compared to Rhenium treatment, thereby indicating no or limited cytotoxic effects from administration of the LHRH compositions (See, FIGS. 5 and 6). Further, the same treated cancer cells show a significant increase in uptake of the LHRH compositions as compared to free Rhenium. This indicates that the LHRH compositions both target and provide enhanced cellular uptake of the nuclides (see, FIGS. 5 and 6).

The LHRH compositions may be administered either alone or in combination with a pharmaceutical carrier and/or a vehicle. Such are understood in the art, see, e.g., Remington: The Science and Practice of Pharmacy, Lippincott Williams & Wilkins, 21st Ed., 2005. The carriers and preparations may include sterile, aqueous or non-aqueous solutions, suspensions, and emulsions. The LHRH compositions may be administered in combination with a slow-release mechanism. Examples of non-aqueous solvents are propylene glycol, polyethylene glycol, vegetable oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The LHRH compositions may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as antimicrobials, anti-oxidants, chelating agents, inert gases, and the like.

The LHRH compositions of the present disclosure may be administered to a patient by any “effective route”. Said effective route may include oral, intrapulmonary, parenteral, subcutaneous, intramuscular, intravenous, intra-arterial, intrathecal, transdermal, and via mucous membrane (nasal, sublingual, rectal, urinary, and reproductive tract). Parenteral infusions can include intraperitoneal administration. Transdermal delivery of the LHRH compositions may be in the form of a slow-release subcutaneous implant. The form may vary depending upon the route of administration. For example, compositions for injection may be provided in the form of an ampoule, each containing a unit dose amount, or in the form of a container containing multiple doses.

The LHRH compositions, in accordance with the present disclosure, may be formulated into diagnostic/therapeutic compositions as pharmaceutically acceptable salts. These salts may include acid addition salts formed with inorganic acids, for example hydrochloric or phosphoric acid, or organic acids, such as acetic oxalic, or tartaric acid and the like. Salts also include those formed from inorganic bases such as sodium, potassium, ammonium, calcium or ferric hydroxides, and organic bases such as isopropylamine, trimethylamine, histidine, procaine and the like. As used herein, an “effective amount” of a compound is an amount that when administered to a patient provides accurate detection of the targeted tumors to a clinically significant degree; or alternatively, to a statistically significant degree as compared to control.

In some aspects, the LHRH compositions may be administered in combination with other therapeutic and/or diagnostic compounds. Further diagnostic compounds may be utilized to co-label a cell to determine if a co-factor or associated protein is present and/or the extent of co-expression with the LHRH receptor. In some aspects, the LHRH compositions may be co-administered with a therapeutic agent. For example, LHRH receptor overexpressssion may be associated with breast and/or endometrial and/or ovarian cancers. As such, co-administration may be a therapeutic agent to treat or alleviate such malignancies and/or tumors is contemplated, including co-administration with raloxifene, tamoxifen, abemaciclib, paclitaxel, everolimus, tastuzumab, alpeli sib, anastrozole, pamidronate, exemestane, atezolizumab, capecitabine, cyclophosphamide, docetaxel, doxorubicin, epirubicin, 5-fluorouracil, toremifene, fluvestrant, letrozole, gemcitabine, eribulin, palbocicib, ixabepilone, pembrolizumab, lapatnib, olaparib, megestrol, methotrexate, neratinib, pertuzumab, ribociclib, sacituzumab, talazoparib, thiotepa, toremifene, tucatinib, vinblastine, melphalan, bevacizumab, carboplatin, cisplatin, topotecan, niraparib, rucaparib, pembrolizumab, lenvatinib, or combinations thereof. Similarly, the LHRH compositions may be useful in detecting or tracking prostate malignancies, abnormalities, tumors and/or cancers in combination with abiraterone, apalutamide, bicalutamide, cabazitaxel, darolutamide, degarelix, docetaxel, enzalutamide, flutamide, mitoxantrone, nilutamide, oalparib, rucaparib, sipuleucel-T, and combinations thereof. The presence of a radionuclide in the LHRH compositions as set forth herein further can provide localized radiation treatment to a cell or region or tissue over-expressing LHRH receptors. For example, the inclusion of radium-223 as a transition or post-transition metal in the LHRH compositions can provide localized radiation treatment, either alone or in combination with other treatments.

In some aspects, the LHRH compositions can be used in the diagnosis of reproductive and non-reproductive cancers expressing the LHRH receptor and/or in monitoring the progression of disease and/or treatment thereof. For example, the use of ^{99m}Tc and ^{186/188}Re-LHRH compositions as set forth herein can increase the concentration of the ^{99m}Tc and ^{186/188}Re nuclides in tumor cells or malignant cells overexpressing LHRH receptors, thereby providing precise and accurate detection. That the LHRH compositions can target cancerous, malignant or tumorigenic cells overexpressing the LHRH receptor with radionuclide compounds provides direct applications to reproductive and non-reproductive cancers and/or malignancies expressing the LHRH receptor, along with, metastatic cancers and rare, hard to treat cancers such as triple negative breast and metastatic ovarian cancer.

In some aspects, the LHRH compositions can target and bind to the LHRH receptors of any cancer cell or any tumor microenvironment that expresses these receptors. Further, the LHRH compositions provide an approach for selective binding that allows for more effective concentrations into the targeted area and cancer cells making them superior conjugates for accurate and quantitative diagnosis. Further, radiopharmaceuticals have applications for cancer therapy as well. See, Tavares et al. Int. J. Radiat. Biol. 2010 (4):261-70. That the LHRH compositions can radionuclides and selectively deliver such to LHRH overexpressing cells provides a selective approach for radiation treatment to those cells and the limited surrounding area.

According to an aspect, either alone or in combination with any other aspect, the present disclosure concerns luteinizing hormone releasing hormone (LHRH) compositions including an LHRH peptide, a linker and a tridentate structure comprised of three amines and a metal or halide nuclide, wherein the three amines retain the metal or halide nuclide.

According to a second aspect, either alone or in combination with any other aspect, the linker can be covalently bound internally to the LHRH peptide.

According to a third aspect, either alone or in combination with any other aspect, the LHRH peptide includes an amino acid sequence as set forth in SEQ ID NO: 2 or SEQ ID NO: 3, wherein X1 is a serine residue, X2 is a tyrosine, phenylalanine, leucine or histidine residue, X3 is glycine, leucine, serine, histidine, lysine, aspartate, glutamate, alanine, or tryptophan residue and X4 is a leucine, valine, tryptophan, or methionine residue.

According to a fourth aspect, either alone or in combination with any other aspect, the linker is covalently bound to X1, X2, X3 or X4.

According to a fifth aspect, either alone or in combination with any other aspect, one or more of X1, X2, X3 or X4 is a right-handed amino acid.

According to a sixth aspect, either alone or in combination with any other aspect, the LHRH peptide is selected from [D-Lys6]-LHRH, leuprorelin, goserelin, buserelin, histrelin, triptorelin, degrelix, nafarelin, [D-Trp6]-LHRH, [D-Ala6]-LHRH, [Gln8]-LHRH, antide, and gonadorelin.

According to a seventh aspect, either alone or in combination with any other aspect, the LHRH peptide includes [D-Lys6]-LHRH.

According to a eighth aspect, either alone or in combination with any other aspect, the linker is covalently bound to the LHRH peptide through a phenyl, an amine, a hydroxyl, a sulfhydryl, a thiol, or a carboxyl group on a side chain of an amino acid therein.

According to a ninth aspect, either alone or in combination with any other aspect, the linker is covalently bound to an amine of a side chain of the LHRH peptide.

According to a tenth aspect, either alone or in combination with any other aspect, the linker includes an amino alkyl carboxylic acid, wherein the alkyl is of between 1 to 25 carbons in length.

According to a eleventh aspect, either alone or in combination with any other aspect, the linker further includes a polyethylene glycol chain of between 1 to 50 repeats in length.

According to a twelfth aspect, either alone or in combination with any other aspect, the tridentate structure is covalently linked to the linker.

According to a thirteenth aspect, either alone or in combination with any other aspect, the tridentate structure is a dialkyltriamine.

According to a fourteenth aspect, either alone or in combination with any other aspect, the tridentate structure is a diethylenetriamine.

According to a fifteenth aspect, either alone or in combination with any other aspect, the metal or halide nuclide is a radionuclide.

According to a sixteenth aspect, either alone or in combination with any other aspect, the radionuclide is selected from ^{99m}technetium, ¹⁸⁶rhenium, ¹⁸⁸rhenium, ^{186/188}rhenium, ¹⁸fluorine, ⁶⁷gallium, ⁶⁸gallium, ⁶⁴copper, ⁶⁷copper, ^{64/67}copper, ⁹⁰yttrium, ¹⁷⁷lutetium, ¹⁹²iridium, ¹⁰³palladium, ⁸⁹strontium, ¹⁵³samarium, ²¹²lead, ²¹³bismuth, ¹³¹caesium, ²²³radium, ²²⁵radium, ²²⁵actinium, ²²⁸thorium, ²²⁷thorium, ²¹¹astatine, and ¹¹¹indium.

According to a seventeenth aspect, either alone or in combination with any other aspect, the radionuclide is ^{99m}technetium.

According to a eighteenth aspect, either alone or in combination with any other aspect, the radionuclide is ^{186/188}rhenium.

According to a nineteenth aspect, either alone or in combination with any other aspect, the LHRH peptide is dimerized through the metal nuclide.

According to a twentieth aspect, either alone or in combination with any other aspect, the LHRH compositions can further include a pharmaceutically acceptable carrier.

According to a twenty-first aspect, either alone or in combination with any other aspect, the present disclosure concerns methods for labeling an LHRH receptor on a cell the administering the LHRH compositions to a cell, whereby the LHRH composition binds the LHRH receptor to provide a label thereof.

According to a twenty-second aspect, either alone or in combination with any other aspect, the cell is in vitro.

According to a twenty-third aspect, either alone or in combination with any other aspect, the cell is from a biopsy or a tissue sample.

According to a twenty-fourth aspect, either alone or in combination with any other aspect, the cell is in vivo.

According to a twenty-fifth aspect, either alone or in combination with any other aspect, the present disclosure concerns methods for determining LHRH receptor expression in a subject comprising administering the LHRH compositions and detecting the metal nuclide thereof.

According to a twenty-sixth aspect, either alone or in combination with any other aspect, the LHRH composition is administered to a subject.

According to a twenty-seventh aspect, either alone or in combination with any other aspect, an LHRH composition includes a structure as set forth in formula I:

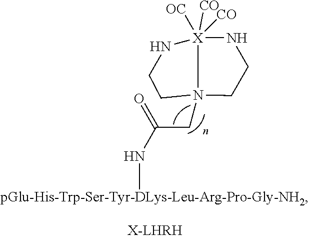

wherein X is a radionuclide and n is from 1 to 25.

According to a twenty-eighth aspect, either alone or in combination with any other aspect, an LHRH composition includes a structure as set forth in formula II:

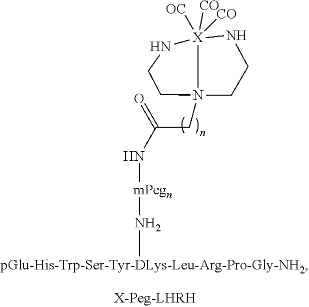

wherein X is a radionuclide and n is from 1 to 25.

According to a twenty-ninth aspect, either alone or in combination with any other aspect, X is selected from a group consisting of rhenium, ^{99m}technetium, ¹⁸⁶rhenium, ¹⁸⁸rhenium, ^{186/188}rhenium, ¹⁸fluorine, ⁶⁷gallium, ⁶⁸gallium, ⁶⁴copper, ⁶⁷copper, ^{64/67}copper, ⁹⁰yttrium, ¹⁷⁷lutetium, ¹⁹²iridium, ¹⁰³palladium, ⁸⁹strontium, ¹⁵³samarium, ²¹²lead, ²¹³bismuth, ¹³¹caesium, ²²³radium, ²²⁵radium, ²²⁵actinium, ²²⁸thorium, ²²⁷thorium, ²¹¹astatine, and ¹¹¹indium.

According to a thirtieth aspect, either alone or in combination with any other aspect, an LHRH composition includes the structure as set forth in Formula III:

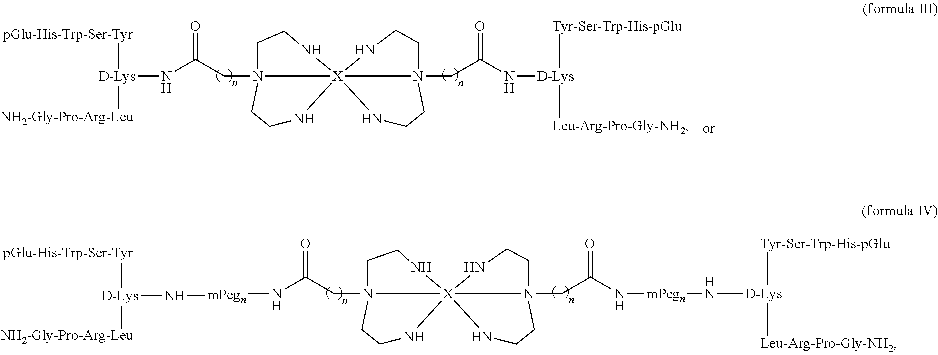

wherein X is a radionuclide and n is from 1 to 25.

According to a thirty-first aspect, either alone or in combination with any other aspect, X is selected from a group consisting of ^{99m}Tc, ^{186/188}Re, ¹¹¹ln, ⁶⁸Ga, ^{64/67}Cu, ⁹⁰Y, and ¹⁷⁷Lu.

According to a thirty-second aspect, either alone or in combination with any other aspect, the present disclosure concerns use of the LHRH composition for the detection and/or identification of an LHRH receptor on a cell.

According to a thirty-third aspect, either alone or in combination with any other aspect, the use of the LHRH composition is on a cell in a subject.

Further aspects and advantages of the disclosure are provided in the following section, which should be considered as illustrative only.

### EXAMPLES

### Example 1

FIG. 1A depicts the synthesis of PEGylated and non-PEGylated M-LHRH using Acdien-LHRH or Acdien-PEG-LHRH with [Re(CO)₃]⁺ or [Tc(CO)₃]⁺ in accordance with a preferred embodiment of the present disclosure. In the preferred example, The Acdien-LHRH and Acdien-PEG-LHRH decapeptides were synthesized using Fmoc solid phase chemistry techniques and then chelated to rhenium or technetium metals. Fmoc-Rink Amide-AM resin was placed onto a reaction vessel and washed with DMF and DCM in continuous-flow mode using a PS3 peptide synthesizer. The side chain protected amino acids derivatives used were Fmoc-Gly-OH, Fmoc-Pro-OH, Fmoc-Arg(Pbf)-OH, Fmoc-Leu-OH, Fmoc-DLys(Aloc)-OH, Fmoc-Tyr(tBu)-OH, Fmoc-Ser(tBu)-OH, Fmoc-Trp(Boc)-OH, Fmoc-His(Trt)-OH and Boc-Glu(OtBu)-OH. The acid labile Boc protecting group is conveniently removed in the end during cleavage of the peptide from the resin. The amino acid couplings employed four equivalents of amino acid and (2-(6-Chloro-1H-benzotriazole-1-yl)-1,1,3,3-tetramethylaminium hexafluorophosphate (HCTU) and sometimes (7-Azabenzotriazol-1-yloxy) tripyrrolidinophosphoniumhexafluorophosphate (PyAOP) or other coupling reagents dissolved in 0.4 M N-methylmorpholine (NMM) in DMF at room temperature. The allyloxycarbonyl (aloc) protecting group on the D-Lys was selectively removed using tetrakis(triphenylphosphine)palladium(0) along with a 37:2:1 mixture of methylene chloride, acetic acid, and NMM for 2 h, followed by washing and double coupling of the Bocdienac linker as described by L. Calderon, et al., Bioconjugate Chemistry, 2017. 28(2): p. 461-470. The amine end of the D-Lys was coupled with either the Bocdienac linker or miniPEG followed by the Bocdienac linker (see FIG. 1A) and the conjugates cleaved from the resin using TFA-Tips-H2O-Phenol or TFA-Tips-H2O or TFA-Thioanisole-H2O or TFA-Thioanisole-H2O cocktail to yield of Acdien-LHRH and Acdien-PEG-LHRH decapeptides respectively.

Complete synthesis of Acdien-PEG-LHRH complex was achieved using the same procedure described for Acdien-LHRH with slight variation. The LHRH peptide was assembled as described by Calderon et al. The Alloc protecting group was selectively removed using palladium as described above and Fmoc-11-amino-3,6,9-trioxaundecanoic acid (mPEG₃) was conjugated to the free amine end. The Fmoc group on mPEG₃ was deprotected with 20% piperidine in DMF for 3 min followed by coupling of the peptide to Bocdienac to give the target peptide. The fully assembled Acdien-PEG-LHRH peptide was cleaved from the solid support using a TFA/water/TIPS cleavage cocktail to give the crude target compound Acdien-PEG-LHRH. Intermediate products were washed between reactions with DMF. Acdien-PEG-LHRH was purified by HPLC or gel filtration using Sephadex G-10. The purified samples were lyophilized before use in the next step. The crude Acdien-PEG-LHRH (see Supporting Information) peptide was purified by HPLC on reverse phase C18 column with a linear gradient from 10% to 90% B eluent in 10 min; tR=6.5 min. The purity of Acdien-peg-LHRH was also confirmed using mass spectrometry. Yield, 31%. ESI-MS (M+H)⁺, calculated for C₇₃H₁₁₄N₂₂O₁₉ 1602.86; found 1602.87.

Acdien-LHRH was dissolved in water, and the pH of the solution was adjusted to 7, by titration with 1 M NaOH. The rhenium complex was prepared in accordance with known methods as described in Lazarova, et al., Inorganic Chemistry Communications 2004, 7 (9), 1023-1026; Schmidt et al., “Manganese(I) and Rhenium(I) Pentacarbonyl(Trifluoromethanesulfonato) Complexes”, In Inorganic Syntheses, John Wiley & Sons, Inc.: 2007; pp 113-117. Equimolar amount of [Re(CO)₃(H₂O)₃]⁺ was reacted with Acdien-LHRH (30 mg, 0.02 mmol) to give the crude Re-Acdien-LHRH conjugate. Re-Acdien-LHRH was purified by HPLC or gel filtration using Sephadex G-10. The purified samples were lyophilized before use in the next step. The crude Re-Acdien-LHRH conjugate was purified by HPLC on reverse phase C18 column with a linear gradient from 10% to 90% B eluent in 10 min; tR=7.3 min. The purity of Re-Acdien-LHRH was confirmed using mass spectrometry.

Complete synthesis of Re-Acdien-PEG-LHRH conjugate was achieved using the same procedure described for Re-Acdien-LHRH. Re-Acdien-PEG-LHRH was purified by HPLC or gel filtration using Sephadex G-10. The purified samples were lyophilized before use in the next step. The crude Re-Acdien-PEG-LHRH was purified by HPLC on a reverse phase C18 column with a linear gradient from 10% to 90% B eluent in 10 min; tR=8.3 min. The purity of Re-Acdien-PEG-LHRH was confirmed using mass spectrometry.

LHRH was labeled with ^{99m}Tc based on a previously reported method. See, Badar et al. EJNMMI Res. 4(1):14-14 (2014). [^{99m}TcO₄]⁻ solution was first prepared with the Isolink kit (Paul Scherrer Institute, Switzerland) according to the manufacturer's instructions to give [^{99m}Tc(CO)₃]⁺, then further reacted with Acdien-LHRH . Typically, 2 mCi ^{99m}TcO₄⁻ was added into one vial of [^{99m}TcO₄]⁻ solution and heated at 100° C. for 20 min using the Isolink kit protocol and reagents. pH was adjusted to about 7-7.4 with HCl. Next, 100 ul of Acdien-LHRH from a stock solution of 1 mg/ml was then added and incubated at 75° C. for 60 min. Afterwards, the labeled peptide was purified by HPLC (C18 column, 10% MeCN+0.1% HOAc 0-10 min, 90% MeCN+0.1% HOAc 10-20 min. 200 nm, 1 ml/min). The retention time was ˜16 min (FIG. 2). Non-decay corrected yield was calculated based on (100*collected radioactivity at 15-17 min)/(total injected radioactivity).

FIG. 1B depicts the synthesis of ^{186/188}Re-Acdien-LHRH using Bocdienac linker by conjugation with the LHRH peptide and [Re(CO)₃(H₂O)₃]Br. The Bocdienac linker was successfully prepared by protection of the primary amines using tert-Butyloxycarbonyl. Once protected, the secondary amine was treated with benzyl 2-bromoacetate to obtain N,N″-bis(tert-butyloxycarbonyl)diethylenetriaminyl-N′-[glycine-benzyl ester] (Bocdienbz). Deprotection of the Z-group in the Bocdienbz ligand was achieved by employing the Pd/C deprotection procedure using H2 gas to give Bocdienac (see FIG. 1B) as described by M. Ndinguri, et al., Inorganica Chimica Acta, 363, 1796-1804 (2010), thereby introducing a carboxylic functional group which was attached to the peptide moiety. Each step was purified by column chromatography or extraction and dried before proceeding to the next step.

### Example 2

FIG. 2 depicts a synthesis of an LHRH-X-LHRH conjugate where X is a radiolabeled transition metal or post-transition metal, such as ^{99m}Tc, ^{186/188}Re, ¹¹¹In, ⁶⁸Ga, ^{64/67}Cu, ⁹⁰Y, or ¹⁷⁷Lu, with or without a PEG group. The Acdien-LHRH and Acdien-pPEGeg-LHRH decapeptides were synthesized as shown in FIG. 1A. Two mole equivalents of each decapeptide are used to react with one mole equivalent of the radionuclide to give LHRH-X-LHRH techniques as described in Notni, et al., EJNMMI Res. 2012 Jun. 9; 2(1):28.

### Example 3

Cell Viability Assay

4T1 mouse mammary tumor and MDA-MB-231 human mammary tumor cell lines were purchased from ATCC. Both cell lines were maintained in Dulbecco's Modified Eagle Medium (DMEM) containing 10% Fetal Bovine Serum, 100 U/ml penicillin and 100 μg/ml streptomycin, incubated at 37° C. in a humidified atmosphere of 95% air and 5% CO₂.

To assess if Re-Acdien-LHRH and Re-Acdien-PEG-LHRH affected cell viability, an MTT Assay was utilized. 4T1 or MDA-MB-231 cells were seeded at 2000 cells/100 μl in a 96-well plate and treated with either [Re(CO)₃(H₂O)₃]⁺, Re-Acdien-LHRH or Re-Acdien-PEG-LHRH from a range of 0.1 μM to 100 μM for 24 hours. Cells were washed 3 times with PBS and incubated in DMEM supplemented with 10% FBS for 48 hours. Afterwards the cells were incubated for 4 hours in 10 μl MTT solution obtained from Vybrant MTT Cell Proliferation Assay Kit (Life Technologies). Lastly, cells were solubilized, mixed with SDS (sodium dodecyl sulfate) and absorbance read at 595 nm on a Phenix Genios Tecon 96 well plate reader. In-vitro assays were used to explore the toxicity of Re-Acdien-LHRH conjugates as an indication for patient safety administration for adverse events. The cellular effect of free Re, Re-Acdien-LHRH, and Re-Acdien-PEG-LHRH conjugates were assayed and compared using methyl thiazol tetrazolium (MTT) (3-(4,5-dimethylthiazol-2-yl)-2,5diphenyltetrazolium bromide) assay as described in S. Aggarwal, et al., International Journal of Cancer, 111 (5), pp. 679-92 (2004).

The results were assayed using MTT (3-(4,5-dimethylthiazol-2-yl)-2,5-diphenyltetrazolium bromide) assay. As shown in FIG. 5 and FIG. 6, both Re-Acdien-LHRH and Re-Acdien-PEG-LHRH did not result in any alteration in normal cell growth compared to Re treatment; indicating cytotoxic effects were not found with administration of the conjugated forms of Re. Re-Acdien-LHRH was not found to significantly inhibit MDA-MB-231 viability compared to the non-LHRH peptide bound Re. Similar results were found with Re-Acdien-PEG-LHRH treatment of MDA-MB-231 and 4T1 cells, in which, no significant attenuation in cell viability was found compared to [Re(CO)₃(H₂O)₃]⁺ treatment (FIGS. 6A and 6B).

### Example 4

Drug Uptake Measurements

To determine cellular uptake of Re-Acdien-LHRH and Re-Acdien-peg-LHRH, 4T1 or MDA-MB-231 cells were seeded 1×10⁶ in 6 well plates and treated with either unbound [Re(CO)₃(H₂O)₃]⁺ (100 μM), Re-Acdien-LHRH (100 μM), or Re-Acdien-PEG-LHRH (100 μM) for 24 hours. Cells were washed 3 times with PBS, harvested, and analyzed for the presence of platinum using inductive coupled plasma (ICP-OES). As shown in FIG. 5 and FIG. 6, there was a significant increase in uptake of the LHRH targeting Re conjugates (Re-Acdien-LHRH and Re-Acdien-PEG-LHRH) compared to free Re in both MDA-MB-231 and 4T1 cell lines (FIGS. 6C and 6D). The 4T1 cell line was used to verify the LHRH targeting results found in the MDA-MB-231 cell line, indicting uptake it is not a cell line specific phenomenon, but the result of receptor presence and expression. This indicates that the new LHRH-radionuclide conjugates are targeting and leading to enhanced cellular uptake of the nuclides.

^{99m}Tc and ^{186/188}Re-LHRH conjugates are projected to be used in clinical settings to target reproductive cancers and any cancer type expressing the LHRH receptor in both animals and humans.

Various identified and recited features are described herein that can be used independently of one another or in combination with other features.

As used herein, the singular forms “a”, “an” and “the” include plural referents unless the context clearly dictates otherwise. “And” as used herein is interchangeably used with “or” unless expressly stated otherwise. As used herein, the term ‘about” means +/−5% of the recited parameter. All embodiments of any aspect of this disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

All the references cited in this disclosure are hereby incorporated by reference in their entirety.

The foregoing description of several aspects of the LHRH compositions and methods of using such have been presented for purposes of illustration. It is not intended to be exhaustive or to limit the application to the precise forms disclosed, and obviously, many modifications and variations are possible in light of the above teaching. It is understood that the disclosure may be applied in ways other than as specifically set forth herein without departing from the scope of the disclosure.

1. A luteinizing hormone releasing hormone (LHRH) composition comprising an LHRH peptide, a linker and a tridentate structure comprised of three amines and a metal or halide nuclide, wherein the three amines retain the metal or halide nuclide.

2. The LHRH composition of claim 1, wherein the linker is covalently bound internally to the LHRH peptide.

3. The LHRH composition of claim 1, wherein the LHRH peptide comprises an amino acid sequence as set forth in SEQ ID NO: 2 or SEQ ID NO: 3, wherein X1 is a serine residue, X2 is a tyrosine, phenylalanine, leucine or histidine residue, X3 is glycine, leucine, serine, histidine, lysine, aspartate, glutamate, alanine, or tryptophan residue and X4 is a leucine, valine, tryptophan, or methionine residue.

4. The LHRH composition of claim 3, wherein the linker is covalently bound to X1, X2, X3 or X4.

5. The LHRH composition of claim 3, wherein one or more of X1, X2, X3 or X4 is a right-handed amino acid.

6. The LHRH composition of claim 1, wherein the LHRH peptide is selected from the group consisting of [D-Lys6]-LHRH, leuprorelin, goserelin, buserelin, histrelin, triptorelin, degrelix, nafarelin, [D-Trp6]-LHRH, [D-Ala6]-LHRH, [Gln8]-LHRH, antide, and gonadorelin.

7. The LHRH composition of claim 1, wherein the LHRH peptide comprises [D-Lys6]-LHRH.

8. The LHRH composition of claim 1, wherein the linker is covalently bound to the LHRH peptide through a phenyl, an amine, a hydroxyl, a sulfhydryl, a thiol, or a carboxyl group on a side chain of an amino acid therein.

9. The LHRH composition of claim 1, wherein the linker is covalently bound to an amine of a side chain of the LHRH peptide.

10. The LHRH composition of claim 1, wherein the linker comprises an amino alkyl carboxylic acid, wherein the alkyl is of between 1 to 25 carbons in length.

11. The LHRH composition of claim 10, wherein the linker further comprises a polyethylene glycol chain of between 1 to 50 repeats in length.

12. The LHRH composition of claim 1, wherein the tridentate structure is covalently linked to the linker.

13. The LHRH composition of claim 1, wherein the tridentate structure is a dialkyltriamine.

14. The LHRH of claim 12, wherein the tridentate structure is a diethylenetriamine.

15. The LHRH composition of claim 1, wherein the metal or halide nuclide is a radionuclide.

16. The LHRH composition of claim 15, wherein the radionuclide is selected from the group consisting of ^{99m}technetium, ¹⁸⁶rhenium, ¹⁸⁸rhenium, ^{186/188}rhenium, ¹⁸fluorine, ⁶⁷gallium, ⁶⁸gallium, ⁶⁴copper, ⁶⁷copper, ^{64/67}copper, ⁹⁰yttrium, ¹⁷⁷lutetium, ¹⁹²iridium, ¹⁰³palladium, ⁸⁹strontium, ¹⁵³samarium, ²¹²lead, ²¹³bismuth, ¹³¹caesium, ²²³radium, ²²⁵radium, ²²⁵actinium, ²²⁸thorium, ²²⁷thorium, ²¹¹astatine, and ¹¹¹indium.

17-18. (canceled)

19. The LHRH composition of claim 1, wherein the LHRH peptide is dimerized through the metal nuclide.

20. (canceled)

21. A method for labeling an LHRH receptor on a cell comprising administering the LHRH composition of claim 1 to a cell, whereby the LHRH composition binds the LHRH receptor to provide a label thereof.

22-24. (canceled)

25. A method for determining LHRH receptor expression in a subject comprising administering the LHRH composition of claim 1 and detecting the metal nuclide thereof.

26. (canceled)

27. The LHRH composition of claim 1 comprising a structure as set forth in formula I: 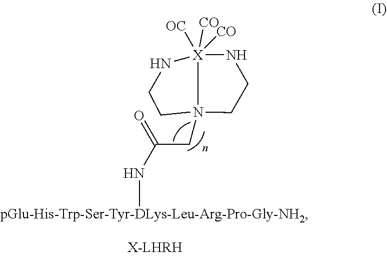
wherein X is a radionuclide and n is from 1 to 25.

28. The LHRH composition of claim 1 comprising a structure as set forth in formula II: 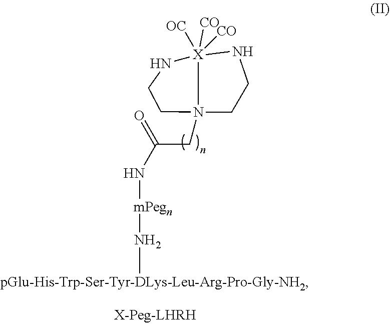
wherein X is a radionuclide and n is from 1 to 25.

29. (canceled)

30. The LHRH composition of claim 19 comprising a structure as set forth in Formula III: 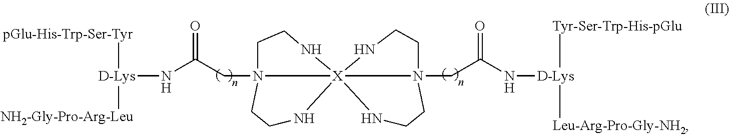
wherein X is a radionuclide and n is from 1 to 25.

31-33. (canceled)


SEQUENCE LISTING (23 sequences)

SEQ ID NO: 1 - protein, 10 aa, Artificial Sequence
  Synthetic Construct.
  MOD_RES (1)..(1): X at position 1 is pyroGlu.
XHWSYGLRPG

SEQ ID NO: 2 - protein, 10 aa, Artificial Sequence
  Synthetic Construct.
  MOD_RES (1)..(1): X at position 1 is pyroGlu.
  MOD_RES (4)..(4): X at position 4 is Ser.
  MOD_RES (5)..(5): X at position 5 is Tyr, Phe, Leu, or His.
  MOD_RES (6)..(6): X at position 6 is Gly, Leu, Ser, His, Lys, Asp, Glu, Ala, or Trp.
  MOD_RES (7)..(7): X at position 7 is Leu, Val, Trp, or Met.
XHWXXXXRPG

SEQ ID NO: 3 - protein, 9 aa, Artificial Sequence
  Synthetic Construct.
  MOD_RES (1)..(1): X at position 1 is pyroGlu.
  MOD_RES (4)..(4): X at position 4 is Ser.
  MOD_RES (5)..(5): X at position 5 is Tyr, Phe, Leu, or His.
  MOD_RES (6)..(6): X at position 6 is Gly, Leu, Ser, His, Lys, Asp, Glu, Ala, or Trp.
  MOD_RES (7)..(7): X at position 7 is Leu, Val, Trp, or Met.
XHWXXXXRP

SEQ ID NO: 4 - protein, 10 aa, Artificial Sequence
  Synthetic Construct.
  MOD_RES (1)..(1): X at position 1 is pyroGlu.
XHWSHGWYPG

SEQ ID NO: 5 - protein, 10 aa, Artificial Sequence
  Synthetic Construct.
  MOD_RES (1)..(1): X at position 1 is pyroGlu.
XHWSHDWKPG

SEQ ID NO: 6 - protein, 10 aa, Artificial Sequence
  Sythetic construct
  MOD_RES (1)..(1): X at position 1 is pyroGlu.
  MOD_RES (6)..(6): X at position 6 is D-Lys.
XHWSYXLRPG

SEQ ID NO: 7 - protein, 9 aa, Artificial Sequence
  Synthetic Construct.
  MOD_RES (1)..(1): X at psoition 1 is pyroGlu.
  MOD_RES (6)..(6): X at psoition 6 is D-Lys.
XHWSYXLRP

SEQ ID NO: 8 - protein, 9 aa, Artificial Sequence
  Synthetic Construct.
  MOD_RES (1)..(1): X at position 1 is pyroGlu.
  MOD_RES (6)..(6): X at position 6 is D-Leu.
  MOD_RES (9)..(9): Pro at position 9 is capped with ethylamide.
XHWSYXLRP

SEQ ID NO: 9 - protein, 9 aa, Artificial Sequence
  Synthetic Construct.
  MOD_RES (1)..(1): X at position 1 is pyroGlu.
  MOD_RES (6)..(6): X at position 6 is D-Ser with appended tertbutyl.
  MOD_RES (9)..(9): Pro at position 9 is capped with hydrazide.
XHWSYXLRP

SEQ ID NO: 10 - protein, 9 aa, Artificial Sequence
  Synthetic Construct.
  MOD_RES (1)..(1): X at position 1 is pyroGlu.
  MOD_RES (6)..(6): X at position 6 is D-Ser appended with tertbutyl.
  MOD_RES (9)..(9): Pro at position 9 is capped with ethylamide.
XHWSYXLRP

SEQ ID NO: 11 - protein, 9 aa, Artificial Sequence
  Synthetic Construct.
  MOD_RES (1)..(1): X at position 1 is pyroGlu.
  MOD_RES (6)..(6): X at position 6 is D-His with appended 1-benzyl.
  MOD_RES (9)..(9): Pro at position 9 is capped with ethylamide.
XHWSYXLRP

SEQ ID NO: 12 - protein, 10 aa, Artificial Sequence
  Synthetic Construct.
  MOD_RES (1)..(1): X at position 1 is pyroGlu.
  MOD_RES (6)..(6): X at position 6 is D-Trp.
XHWSYXLRPG

SEQ ID NO: 13 - protein, 10 aa, Artificial Sequence
  Synthetic Construct.
  MOD_RES (1)..(1): X at position 1 is acetly-D-Ala with appended 2-naphthyl.
  MOD_RES (2)..(2): X at position 2 is D-Phe with appended 4-chloro.
  MOD_RES (3)..(3): X at position 3 is D-Ala with appended 3-pyridyl.
  MOD_RES (5)..(5): Phe at position 5 is appended 4-S-dihydroorotamido.
  MOD_RES (6)..(6): X at position 6 is D-Phe with appended 4-ureido.
  MOD_RES (8)..(8): Lys at position 8 is appended isopropyl.
  MOD_RES (10)..(10): X at position 10 is D-Ala.
XXXSFXLYPX

SEQ ID NO: 14 - protein, 10 aa, Artificial Sequence
  Synthetic Construct.
  MOD_RES (1)..(1): X at position 1 is pyroGlu.
  MOD_RES (6)..(6): X at position 6 is D-Ala with appended 2-naphthyl.
XHWSYXLRPG

SEQ ID NO: 15 - protein, 10 aa, Artificial Sequence
  Synthetic Construct.
  MOD_RES (1)..(1): X at position 1 is pyroGlu.
  MOD_RES (6)..(6): X at position 6 is D-Trp.
XHWSYXLRPG

SEQ ID NO: 16 - protein, 10 aa, Artificial Sequence
  Synthetic Construct.
  MOD_RES (1)..(1): X at position 1 is pyroGlu.
  MOD_RES (6)..(6): X at position 6 is D-Ala.
XHWSYXLRPG

SEQ ID NO: 17 - protein, 10 aa, Artificial Sequence
  Synthetic Construct.
  MOD_RES (1)..(1): X at position 1 is pyroGlu.
XHWSYGLQPG

SEQ ID NO: 18 - protein, 10 aa, Artificial Sequence
  Synthetic Construct.
  MOD_RES (1)..(1): X at position 1 is acetyl-3-(2-naphthyl)-D- alanine.
  MOD_RES (2)..(2): X at position 2 is D-Phe with appended 4-chloro.
  MOD_RES (3)..(3): X at position 3 is D-(3-pyridyl)alanine.
  MOD_RES (5)..(5): Lys at position 5 is appended with nicotinyl
  MOD_RES (6)..(6): X at position 6 is D-Lys appended with nicotinyl.
  MOD_RES (8)..(8): Lys at position 8 is appended with isopropyl.
  MOD_RES (10)..(10): X at position 10 is D-Ala.
XXXSYSLYPX

SEQ ID NO: 19 - protein, 10 aa, Artificial Sequence
  Synthetic Construct.
  MOD_RES (1)..(1): X at position 1 is pyroGlu.
XHWSYGLRPG

SEQ ID NO: 20 - protein, 10 aa, Artificial Sequence
  Synthetic Construct.
  MOD_RES (4)..(4): X at position 4 is Ser.
  MOD_RES (5)..(5): X at position 5 is Tyr, Phe, Leu, or His.
  MOD_RES (6)..(6): X at position 6 is Gly, Leu, Ser, His, Lys, Asp, Glu, Ala, or Trp.
  MOD_RES (7)..(7): X at position 7 is Leu, Val, Trp, or Met.
EHWXXXXRPG

SEQ ID NO: 21 - protein, 9 aa, Artificial Sequence
  Synthetic Construct.
  MOD_RES (4)..(4): X at position 4 is Ser.
  MOD_RES (5)..(5): X at position 5 is Tyr, Phe, Leu, or His.
  MOD_RES (6)..(6): X at position 6 is Gly, Leu,Ser, His, Lys, Asp, Glu, Ala, or Trp.
  MOD_RES (7)..(7): X at position 7 is Leu, Val, Trp, or Met.
EHQXXXXRP

SEQ ID NO: 22 - protein, 10 aa, Artificial Sequence
  Synthetic Construct.
EHWSHGWYPG

SEQ ID NO: 23 - protein, 10 aa, Artificial Sequence
  Synthetic Construct.
EHWSHDWKPG